(19) United States
(12) Patent Application Publication
(10) Pub. No.: US 2026/0144729 A1
(43) Pub. Date: May 28, 2026

(54) A CLEANSING COMPOSITION

(71) Applicant: L'OREAL, Paris (FR)

(72) Inventors: Jie DENG, Shanghai (CN); Xiuxia WANG, Shanghai (CN); Ning CHANG, Shanghai (CN)

(73) Assignee: L'OREAL, Paris (FR)

(21) Appl. No.: 19/121,168

(22) PCT Filed: Oct. 31, 2022
(86) PCT No.: PCT/CN2022/128540

§ 371 Date: Apr. 15, 2025

(51) Int. Cl. A61K 8/46; A61Q 5/02; A61Q 19/10

(52) U.S. Cl. CPC A61K 8/466; A61Q 5/02; A61Q 19/10; A61K 2800/596

(57) ABSTRACT

It relates to a cleansing composition, comprising at least one sulfur-containing anionic surfactant, at least one betaine-derive amphoteric surfactant from sultains, at least a-hydroxy acid, at least one β-hydroxy acid; and at least one organic basifying agent selected from alkanolamines.

### TECHNICAL FIELD

The present invention relates to a cleansing composition, especially one providing long-lasting clean effect to keratin material, such as hair and scalp.

### BACKGROUND

Skin is present widely on surface of human bodies. Many people have for a long time sought to fresh the body surface, so as to feel comfortable in daily life and work. It has always been an ultimate target of the cosmetic filed to deliver products with skin benefits such as hydration, moisturizing, anti-aging, whitening, cleansing, slippery feeling and so on.

Greasy scalp and hair are becoming the annoying scalp issue worldwide; people usually use oil control shampoo to remove dirt and sebum on scalp. But consumers are not satisfied with the long-lasting clean between two hair washes. Current normal shampoo contains surfactants as the cleansing agent, which aims generally at removing liquid sebum just secreted. Liquid sebum composition will be decomposed by fungi and bacterial on scalp and form stubborn sebum together with dead cells. The ability of normal shampoo to remove the stubborn sebum is still to be enhanced for efficient cleansing of greasy scalp. The use of surfactants in the cleansing products are thus to be improved.

Acids are widely used in cosmetics. For example, acid can be functional on unlock the SC cell desmosomes which is a key part for cells connection. Specifically, acid, such as alpha hydroxy acid or beta hydroxy acid, helps to remove the connection structure between the keratin in the stratum corneum, and shows a gentle melting effect of the keratin. However, the PH of the composition gets lower with the adding the acid results in decreasing of the amount of foam.

There is thus still needed in the art to develop new compositions suitable with desirable cleansing properties for greasy keratin material, such as scalp and hair, and with abundant, stable and soft foam.

### SUMMARY OF THE INVENTION

By deep study, the inventors have surprisingly discovered that by using specifically anionic surfactant, amphoteric surfactant, hydroxy acids and organic basifying agent, the composition can provide improved cleansing property, e.g., long-lasting clean effect, to keratin material, such as hair and scalp and with abundant, stable and soft foam.

Thus, one subject of the present invention is a composition, comprising:
A) at least one sulfur-containing anionic surfactant;
B) at least one betaine-derived amphoteric surfactant selected from sultains;
C) at least one α-hydroxy acid;
D) at least one β-hydroxy acid;
E) at least one organic basifying agent selected from alkanolamines.

The composition according to the present invention is particularly useful for products where a long-lasting clean effect is desirable, in particular for a hair and scalp product. Accordingly, the composition of the present invention can be particularly used in a cleansing product for hair and scalp, e.g., a shampoo providing abundant and stable foam.

### EMBODIMENTS OF THE INVENTION

Throughout the description, including the claims, the term “comprising a” should be understood as being synonymous with “comprising at least one”, unless otherwise mentioned. Moreover, the expression “at least one” used in the present description is equivalent to the expression “one or more”.

Throughout the description, including the claims, an embodiment defined with “comprising” or the like should be understood to encompass a preferable embodiment defined with “consisting substantially of” and a preferable embodiment defined with “consisting of”.

Other than in the operating examples, or where otherwise indicated, all numbers expressing quantities of components and/or reaction conditions are to be understood as being modified in all instances by the term “about”, with conventionally known meaning in the art, e.g., within 10% of the indicated number (e.g. “about 10%” means 9%-11% and “about 2%” means 1.8%-2.2%).

In the application, unless specifically mentioned otherwise, contents, parts and percentages are expressed on a weight basis.

The present invention is directed to a composition comprising:
A) at least one sulfur-containing anionic surfactant;
B) at least one betaine-derived amphoteric surfactant selected from sultains;
C) at least one α-hydroxy acid;
D) at least one β-hydroxy acid;
E) at least one organic basifying agent selected from alkanolamines.

The present invention thus provides use of the composition according to the present invention for providing long-lasting clean effect, e.g., providing abundant and stable foam.

Other characteristics and advantages of the invention will emerge more clearly on reading the description and the examples that follow.

### Component A), Sulfur-Containing Anionic Surfactant

According to an embodiment of the invention, the composition according to the present invention comprises at least one sulfur-containing anionic surfactant, for use as component A).

The term “anionic surfactant” is understood to mean an amphiphilic compound with a hydrophobic part and a hydrophilic part wherein the hydrophilic part carries as ionic or ionisable group only anionic group with a cationic counterion which is generally metallic (alkali metal, such as Na or K) or ammonium, capable of dissociating to give anions in aqueous solution.

For the purpose of the present invention, the term “sulfur-containing anionic surfactant” is understood to mean an anionic surfactant comprising at least one sulfur atom in the molecules.

Accordingly, the anionic group of the anionic surfactant is particularly belonging to the group chosen from: —SO₃H, —S(O)₂O⁻, —OS(O)₂OH, -and OS(O)₂O⁻, the cationic counter anion being usually selected from alkali metal such as sodium, or alkaline earth metal such as magnesium, or organic cationic counter anion such as ammonium salts, amine salts, or aminoalcohol salts. The surfactants may also occur in their acid forms.

Mention may be made, as sulfur-containing anionic surfactants, of surfactants comprising sulfate, sulfonate, sulfoacetate, or sulfosuccinate, and their mixtures.

More particularly, the anionic surfactant according to the invention is chosen from:
(C₆-C₃₀) alkyl sulfates, (C₆-C₃₀) alkyl ether sulfates, (C₆-C₃₀) alkylamido ether sulfates, alkylaryl polyether sulfates or monoglyceride sulfates; preferably for this type of anionic surfactants, (C₆-C₃₀) alkyl ether sulfates, alkylaryl polyether sulfates, or a mixture is used. Mentions may be made of sulfate of ether of lauryl alcohol and alkylene oxide, containing from 1 to 50 alkylene oxide groups.
(C₆-C₃₀) alkyl sulfonates, (C₆-C₃₀) alkylamidesulfonates, (C₆-C₃₀) alkylaryl sulfonates, α-olefin sulfonates, paraffin sulfonates;
(C₆-C₃₀) alkyl sulfosuccinates, (C₆-C₃₀) alkyl ether sulfosuccinates or (C₆-C₃₀) alkylamido sulfosuccinates;
(C₆-C₃₀) alkyl sulfoacetates;
(C₆-C₃₀) alkylpolyglycoside sulfosuccinates;
(C₆-C₃₀) alkyl sulfosuccinamates;
and their mixtures.

The alkyl or acyl radicals of these various anionic surfactants preferably comprise from 12 to 20 carbon atoms.

Preferably, the sulfur-containing anionic surfactant is selected from the group consisting of sulfate, sulfonate, salts of fatty acids, salts of galactosiduronic acids, salts of ether carboxylic acids and their mixtures.

According to an embodiment of the present invention, the anionic surfactant is preferably chosen from sodium laureth sulfate, sodium lauroyl methyl isethionate, sodium cocoyl isethionate, or a mixture thereof.

According to an embodiment of the present invention, the anionic surfactant is sodium laureth sulfate.

The component A) may be present in the composition according to the present invention in an amount ranging from 1% to 50%, such as from 5% to 40%, or from 10% to 30%, by weight, relative to the total weight of the composition.

Component B), Amphoteric Surfactant Selected from Sultains

According to an embodiment of the invention, the composition according to the present invention comprises at least one amphoteric surfactant, which may also be called as zwitterionic surfactant, which is preferably selected from sultains, for use as component B).

The amphoteric or zwitterionic surfactant(s) may be a betaine surfactant, e.g., (C₈-C₂₀) alkylbetaines, sulfobetaines (also called sultaines), (C₈-C₂₀ alkyl) amido (C₂-C₈ alkyl) betaines and (C₈-C₂₀ alkyl) amido (C₂-C₈ alkyl) sulfobetaines.

As (C8-C20) alkylbetaines, mention may notably be made of cocobetain like the product marketed under the name of DEHYTON AB-30® by Cognis, laurylbetain like the product marketed under the name of GENAGEN KB® by Clariant, oxyethylene laurylbetain (10 EO), like the product marketed under the name of LAURYLETHER (10 EO) BETAIN® by Shin Nihon Rica, oxyethylene stearylbetain (10 EO) like the product marketed under the name of STEARYLETHER (10 EO) BETAIN® by Shin Nihon Rica.

Among the (C8-C20) alkylamido (C1-C6) alkylbetaines, mention may for example be made of cocamidopropyl betaine like the products marketed under the name of LEBON 2000 HG® by Sanyo, under the name of EMPIGEN BB® by Albright & Wilson, under the names of Tego Betain F 50 and CK D by EVONIK GOLDSCHMIDT, or further those marketed as a mixture with glyceryl laurate like the commercial references Tego Betain HS or Antil HS 60 from EVONIK GOLDSCHMIDT, lauramidopropyl betaine like the product marketed under the name of REWOTERIC AMB12P® by Witco. For example, (C8-C20) alkylamido (C1-C6) alkylbetaines may be chosen among (C8-C20) alkylamidopropylbetaines.

For the purpose of the present invention, sultains are particularly preferred for use as component B). Mention may be made of (C8-C₂₀) alkylsulfobetaines; alkyl (C8-C20) amidoalkyl (C1-C6) sulfobetaines, alkyl (C8-C20) amidoalkyl (C1-C6) hydroxyl sulfobetaines, such as cocoyl-amidopropylhydroxy-sulfobetaine, such as the one marketed under the name of CROSULTAIN C-50® by Croda.

Among all the amphoteric surfactants mentioned above, use is preferably made of sultains. That is, for the purpose of present invention, according to an embodiment, both the anionic surfactant of component A) and the amphoteric surfactant of component B) may comprise sulfur atom in the molecule. Without being limited by any known theory, it is believed that for a cleansing product, especially a foamable one, the combination use of components A) and B) can particularly benefit the foaming performance, e.g., the abundance and/or stability of the foam.

The component B) may be present in the composition according to the present invention in an amount ranging from 0.1% to 20% by weight, preferably from 0.3% to 12% by weight, or preferably from 0.5% to 8% by weight, relative to the weight of the composition.

For the purpose of the present invention, components A) and B) are concurrently used in the composition of the invention. According to an embodiment of the present invention, the combination of components A) and B) may be present in the composition according to the present invention in an amount ranging from 1.1% to 60% by weight, preferably from 5% to 40% by weight, or preferably from 10% to 30% by weight, relative to the weight of the composition.

### Hydroxy Acids

Acids are widely used in cosmetic products, including use as chelating agents, preservatives, pH-adjusting agents, active components, and the like.

For a cleansing product, the basic performance is the cleansing effect naturally; and for hair and/or scalp product, e.g., a shampoo, the removing performance of sebum is highly interested. The inventors discover that the combination of specific hydroxy acids is incorporated, the cleansing effect can be desirable.

### Component C), α-Hydroxy Acid

According to an embodiment of the invention, the composition according to the present invention comprises at least one alpha hydroxy acid, for use as component C).

The term “α-hydroxy acid” is understood to mean, according to the present invention, a carboxylic acid having at least one hydroxyl functional group occupying an α-position on said acid (carbon adjacent to a carboxylic acid functional group).

The α-hydroxy acids (α-hydroxy acid) include, for example, citric acid, lactic acid, methyllactic acid, glucuronic acid, glycolic acid, pyruvic acid, 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytetradecanoic acid, 2-hydroxyhexadecanoic acid, 2-hydroxyoctadecanoic acid, 2-hydroxytetracosanoic acid, 2-hydroxyeicosanoic acid, mandelic acid, phenyllactic acid, gluconic acid, galacturonic acid, aleuritic acid, ribonic acid, tartronic acid, tartaric acid, malic acid, fumaric acid and their mixtures. It is also possible to use mixtures of these various acids.

According to an embodiment of the present invention, the α-hydroxy acid is preferably a lower acid having 2-12 carbon atoms, or 2-8 carbon atoms, or 2-6 carbon atoms.

According to an embodiment of the present invention, the α-hydroxy acid is preferably a mono-carboxylic acid.

According to a preferable embodiment of the present invention, the α-hydroxy acid is a lower mono-carboxylic acid having 2-6 carbon atoms, such as lactic acid, gluconic acid, or glycolic acid.

The component C) may be present in the composition according to the present invention in an amount ranging from 0.1 to 40% by weight, from 0.5 to 20% by weight, or from 1.5 to 10% by weight, relative to the total weight of the composition.

### Component D), β-Hydroxy Acid

According to an embodiment of the invention, the composition according to the present invention comprises at least one β-hydroxy acid, for use as component D).

The term “β-hydroxy acid” is understood to mean, according to the present invention, a carboxylic acid that having a carboxylic acid functional group and hydroxy functional group separated by two carbon atoms.

Suitable β-hydroxy acids includes salicylic acid, propionic acid, β-hydroybutyric acid, β-hydroxy β-methylbutyric acid, carnitine, derivatives thereof, and combinations thereof.

Preferably, the β-hydroxy acid is selected from salicylic acid and its derivative of the formula:

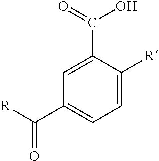

wherein R is a linear, branched or cyclic saturated aliphatic group or an aliphatic unsaturated group containing one or a number of double bonds, which may or may not be conjugated, these groups containing from 2 to 22, preferably 3 to 11 carbon atoms and being able to be substituted for example by at least one substituent selected from (a) halogen atoms, (b) the trifluoromethyl group, (c) hydroxyl groups in the free form or esterified by an acid having from 1 to 6 carbon atoms or (d) a carboxyl functional group which is free or esterified by a lower alcohol having from 1 to 6 carbon atoms;

R′ is a hydroxyl group or an ester functional group of the following formula:

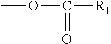

wherein R₁ is a linear or branched saturated or unsaturated aliphatic group having from 1 to 18 carbon atoms.

According to a preferable embodiment of the present invention, the β-hydroxy acid may be preferably one having a salicylic moiety, including: salicylic acid, acetylsalicylic acid, capryloyl salicylic acid, 5-n-decanoylsalicylic acid, 5-n-dodecanoylsalicylic acid, 5-n-octylsalicylic acid, 5-n-heptyloxysalicylic acid, 4-n-heptyloxysalicylic acid, 5-tert-octylsalicylic acid, 3-tert-butyl-5-methylsalicylic acid, 3-tert-butyl-6-methylsalicylic acid, 3,5-diisopropylsalicylic acid, 5-butoxysalicylic acid, 5-octyloxysalicylic acid, 5-propanoylsalicylic acid, 5-n-hexadecanoylsalicylic acid, 5-n-oleoylsalicylic acid, 5-benzoylsalicylic acid, monovalent and divalent salts thereof, and mixtures thereof. Preference may be made to, e.g., salicylic acid, acetylsalicylic acid, and capryloyl salicylic acid.

Non-limiting examples of other useful β-hydroxy acids include β-hydroxypropionic acid, β-hydroxybutyric acid, β-hydroxy β-methylbutyric acid, carnitine, lipohydroxy acid, 3-hydroxy valeric acid, and a mixture thereof.

The component D) may be present in the composition according to the present invention in an amount ranging from 0.1 to 30% by weight, from 0.5 to 15% by weight, or from 1 to 8% by weight, relative to the total weight of the composition.

### Component E), Organic Basifying Agent

The composition according to the present invention may comprise at least one pH adjusting agent (pH adjuster). As the pH adjusting agent, at least one acidifying agent and/or at least one basifying agent (alkaline agent) may be used, preferably a basifying agent (alkaline agent).

The basifying agent may be a monovalent or polyvalent, such as divalent, base.

The basifying agents may be mineral (inorganic) or organic, or hybrid.

The mineral basifying agents may be chosen from aqueous ammonia; alkali metal carbonates or bicarbonates such as sodium or potassium carbonates and sodium or potassium bicarbonates; alkali metal hydroxides such as sodium hydroxide and potassium hydroxide; and mixtures thereof.

Amongst others, the organic basifying agent is to be preferably used as component E), in addition to and concurrently with the components A) to D), for the purpose of the invention. The organic basifying agents may be chosen from organic amines with a pKb at 25° C. of less than 12, preferably less than 10, and even more advantageously less than 6. It should be noted that it is the pKb corresponding to the function of highest basicity. In addition, the organic amines do not comprise any alkyl or alkenyl fatty chains comprising more than ten carbon atoms.

The organic basifying agent may be chosen, for example, from alkanolamines, oxyethylenated and/or oxypropylenated ethylenediamines, amino acids and amine compounds of formula (III) below:

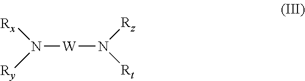

in which
   W represents a C₁-C₆ divalent alkylene radical optionally substituted with one or more hydroxyl groups or a C₁-C₆ alkyl radical, and optionally interrupted with one or more heteroatoms such as O and N, and
   Rₓ, R_{y}, R_{z}, and Rₜ, which may be identical or different, represent a hydrogen atom or a C₁-C₆ alkyl, C₁-C₆ hydroxyalkyl, or C₁-C₆ aminoalkyl radical.

Examples of the amine compounds of formula (III) that may be mentioned include 1,3-diaminopropane, 1,3-diamino-2-propanol, spermine and spermidine.

Alkanolamine is preferably used as component E). The term “alkanolamine” means an organic amine comprising a primary, secondary or tertiary amine function, and one or more linear or branched C₁-C₈ alkyl groups bearing one or more hydroxyl radicals.

Alkanolamines such as monoalkanolamines, dialkanolamines or trialkanolamines comprising one to three identical or different C₁-C₄ hydroxyalkyl radicals may be suitable for the present invention. Among the compounds of this type, mention may be made of monoethanolamine (MEA), diethanolamine, triethanolamine, monoisopropanolamine, diisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol and tris(hydroxymethylamino) methane.

In a preferred embodiment of the present invention, the organic basifying agent may be selected from alkanolamines, in particular monoethanolamine, diethanolamine, or triethanolamine. Even more preferentially, the organic basifying agent may be triethanolamine.

The pH adjusting agent, e.g., the organic basifying agent, may be present in an amount of 0.01% by weight or more, preferably 0.05% by weight or more, and more preferably 0.1% by weight or more, relative to the total weight of the composition.

The pH adjusting agent, e.g., the organic basifying agent, may be present in an amount of 15% by weight or less, preferably 10% by weight or less, and more preferably 5% by weight or less, relative to the total weight of the composition.

The pH adjusting agent, e.g., the organic basifying agent, may be present in an amount ranging from 0.01% to 15% by weight, preferably from 0.05% to 10% by weight, and more preferably from 0.1% to 5% by weight or less, relative to the total weight of the composition.

It is preferable that the composition according to the present invention have a pH of 2.5 or more, and more preferably 3 or more.

It is preferable that the composition according to the present invention have a pH of 6.5 or less, and more preferably 5 or less.

It is preferable that the composition according to the present invention have a pH of from 2.5 to 6.5, and more preferably from 3.5 to 4.5.

The pH of the composition means the pH of the aqueous phase of the composition according to the present invention.

According to an embodiment of the present invention, at least one buffer or buffering agent may also be used, in combination with the acidifying agent and/or the basifying agent, in order to stabilize the pH of the composition according to the present invention.

As the buffer, any of commonly known buffers may be used. For example, salts of acids or bases, preferably salts of weak acids or weak bases, may be used. For example, sodium citrate or sodium lactate may be used as the buffer, if citric acid or lactic acid is used as the acidifying agent. The buffer, if any, is used in an amount being capable of stabilizing the pH in the ranges desired above.

### Component F), Nonionic Surfactant

The composition according to the present invention may preferably comprise at least one nonionic surfactant, for use as component F), which may be a conventional surfactant or particularly one derived from sugar.

Examples of the useful nonionic surfactants may comprise esters of polyols and of fatty acids with a saturated or unsaturated chain containing, for example, from 8 to 24 carbon atoms and better still from 12 to 22 carbon atoms, and the oxyalkylenated derivatives thereof, i.e, derivatives containing oxyethylenated and/or oxypropylenated units, e.g., oxyalkylenated derivatives thereof; the polyethylene glycol esters of C₈-C₂₄ fatty acids, and the oxyalkylenated derivatives thereof; the sorbitol esters of C₈-C₂₄ fatty acids, and the oxyalkylenated derivatives thereof; the sugar (sucrose, glucose or alkylglucose) esters of fatty acids, in particular C₈-C₂₄ fatty acids (fatty acid esters of glucose or of alkylglucose), and the oxyalkylenated derivatives thereof; fatty alcohol ethers; the sugar ethers of fatty alcohols, in particular C₈-C₂₄ fatty alcohols, and mixtures thereof.

Sugar esters of fatty acid can be particularly fatty acid esters of glucose or of alkylglucose, which may be mentioned in particular include glucose palmitate, alkylglucose sesquistearates, for instance methylglucose sesquistearate, alkylglucose palmitates, for instance methylglucose palmitate or ethylglucose palmitate, fatty esters of methylglucoside and more especially the diester of methylglucoside and of oleic acid (CTFA name: methyl glucose dioleate); the mixed ester of methylglucoside and of the acid/hydroxystearic acid (CTFA oleic mixture name: methyl glucose dioleate/hydroxysterate); the ester of methylglucoside and of isostearic acid (CTFA name: methyl glucose isostearate); the ester of methylglucoside and of lauric acid (CTFA name: methyl glucose laurate); the mixture of the monoester and diester of methylglucoside and of isostearic acid (CTFA name: methyl glucose sesquiisostearate); the mixture of the monoester and diester of methylglucoside and of stearic acid (CTFA name: methyl glucose sesquistearate) and in particular the product sold under the name Glucate® SS by the company Amerchol, and mixtures thereof.

Examples of oxyethylenated ethers of a fatty acid and of glucose or of alkylglucose that may be mentioned include the oxyethylenated ethers of a fatty acid and of methylglucose, and in particular the polyethylene glycol ether of the diester of methyl glucose and of stearic acid containing about 20 mol of ethylene oxide (CTFA name: PEG-20 methyl glucose distearate), such as the product sold under the name Glucam R E-20 distearate by the company Amerchol; the polyethylene glycol ether of the mixture of monoester and diester of methylglucose and of stearic acid containing about 20 mol of ethylene oxide (CTFA name: PEG-20 methyl glucose sesquistearate) and in particular the product sold under the name Glucamate® SSE-20 by the company Amerchol, and the product sold under the name Grillocose® PSE-20 by the company Goldschmidt, and mixtures thereof.

Examples of sucrose esters that may be mentioned include sucrose palmitostearate, sucrose stearate and sucrose monolaurate.

Examples of fatty alcohol ethers that may be mentioned include polyethylene glycol ethers of fatty alcohols containing from 8 to 30 carbon atoms and especially from 10 to 22 carbon atoms, such as polyethylene glycol ethers of cetyl alcohol, of stearyl alcohol or of cetearyl alcohol (mixture of cetyl alcohol and stearyl alcohol). Examples that may be mentioned include ethers comprising from 1 to 200 and preferably from 2 to 100 oxyethylene groups, such as those of CTFA name Ceteareth-20 and Ceteareth-30, and mixtures thereof.

Sugar ethers that may especially be mentioned are alkylpolyglucosides, for example decylglucoside, for instance the product sold under the name Mydol® 10 by the company Kao Chemicals, the product sold under the name Plantaren® 2000 by the company Henkel, and the product sold under the name Oramix® NS 10 by the company SEPPIC; caprylyl/capryl glucoside, for instance the product sold under the name Oramix® CG 110 by the company SEPPIC or under the name Lutensol® GD 70 by the company BASF; laurylglucoside, for instance the products sold under the names Plantaren® 1200 N and Plantacare® 1200 by the company Henkel; cocoglucoside, for instance the product sold under the name Plantacare 818/UP by the company Henkel; cetostearyl glucoside optionally as a mixture with cetostearyl alcohol, sold, for example, under the name Montanov® 68 by the company SEPPIC, under the name Tego-Care® CG90 by the company Goldschmidt and under the name Emulgade® KE3302 by the company Henkel; arachidyl glucoside, for example in the form of the mixture of arachidyl alcohol and behenyl alcohol and arachidyl glucoside, sold under the name Montanov® 202 by the company SEPPIC; cocoylethylglucoside, for example in the form of the mixture (35/65) with cetyl alcohol and stearyl alcohol, sold under the name Montanov® 82 by the company SEPPIC; and mixtures thereof.

Amongst others, examples can be particularly made to the sugar esters of fatty acids or the oxyalkylenated derivatives thereof, and the sugar ethers of fatty alcohols, and mixtures thereof. In particular, the sugar ethers of fatty alcohols, in particular C8-C24 fatty alcohols, can be preferred.

The component F) may be present in the composition according to the present invention in an amount ranging from 0.1% to 20% by weight, such as from 0.5% to 10% by weight, or from 1% to 5% by weight, relative to the total weight of the composition.

For the purpose of the present invention, especially for the foamability desired, the concurrent use of component F) in combination with components A) and B) in the composition of the present invention may be preferable. According to an embodiment of the present invention, the combination of components A), B) and F) may be present in the composition according to the present invention in an amount ranging from 1.2% to 60% by weight, preferably from 5% to 45% by weight, or preferably from 12% to 30% by weight, relative to the weight of the composition.

When component F) is used in combination with components A) and B) in the composition of the invention, the use of component E) of the organic basifying agent in the composition can still benefit the purpose of the invention.

### Additional Active Ingredient

The composition according to the invention may advantageously comprise one or additional active ingredients, in particular cosmetic, dermatological or pharmaceutical.

In the context of the present invention, an additional active ingredient is an active ingredient other than the α- or β-hydroxy acids present in the composition and the other active agents specifically defined above.

Advantageously, such additional active ingredient, cosmetic, dermatological or pharmaceutical, may be intended to exert a cosmetic, care or hygiene effect on keratin materials such as skin, including face, hair, eyelashes, scalp and/or leather hairy, and preferentially on the hair or scalp.

The choice of this additional active ingredient is of course conditioned by the effect sought jointly by the composition in question.

Of course, those skilled in the art will take care to choose the optional additional active ingredient(s) added to the composition according to the invention in such a way that the advantageous properties intrinsically attached to the composition according to the invention are not, or substantially not, altered by the proposed addition.

These additional active ingredients are generally present in the composition in a content ranging from 0.0001% to 20% by weight and preferably from 0.01% to 10% by weight relative to the total weight of said composition.

### Additives

According to various embodiments, the compositions of the present invention are provided for application to keratin materials. In accordance with these embodiments, the compositions of the present invention can comprise various ingredients conventionally useful in compositions for conditioning keratin materials, such as, humectants, fatty substances, antidandruff agents, anti-seborrhoea agents, vitamins and provitamins including panthenol, sequestrants, antioxidants, hydroxyacids, fragrances and preserving agents.

A non-exhaustive listing of such ingredients can be found in U.S. patent application publication No. 2004/0170586, the entire contents of which is hereby incorporated by reference.

A person skilled in the art will take care to select the optional additional additives and/or the amount thereof such that the advantageous properties of the composition according to the invention are not, or are not substantially, adversely affected by the envisaged addition.

These additives may be selected variously by the person skilled in the art in order to prepare a composition which has the desired properties, for example, consistency or texture. In particular, the additives, if used, and the amounts thereof are particularly determined according to the specific products/applications thereof, e.g., la shampoo and the like.

These additives may be present in the composition in an amount from 0.01% to 50% by weight relative to the total weight of the composition, including all ranges and subranges therebetween.

### Method and Use

The composition according to the present invention can be generally prepared according to the general knowledge of a person skilled in the art. Nevertheless, it is to be understood that a person skilled in the art can choose the method of preparation, on the basis of his/her general knowledge, taking into account the nature of the constituents used, for example, their solubility in the vehicle, and the application envisaged for the compositions or the kit.

According to an embodiment, the composition according to the present invention can be used to formulate products useful for cosmetic use, e.g., for cleansing keratin materials.

The composition according to the present invention is preferably useful to formulate cleansing products, especially for hair and/or scalp. The present invention thus provides use of a combination of specific surfactants of components A) and B), or of components A), B) and F), for enhancing the foaming performance of the combination of specific hydroxy acids of components C) and D) according to the present invention.

The invention will be further illustrated by the following examples, which set forth particularly advantageous embodiments.

Notwithstanding that the numerical ranges and parameters setting forth the broad scope of the present invention are approximations, the numerical values set forth in the specific examples are reported as precisely as possible. Any numerical value, however, inherently contain certain errors necessarily resulting from the standard deviation found in their respective measurements. The following examples are intended to illustrate the present invention without limiting the scope as a result.

### EXAMPLES

The ingredient amounts/concentrations in the compositions/formulas described below were expressed in % by weight, relative to the total weight of each composition/formula.

Main raw materials used, trade names and supplier thereof are listed below. Materials without specification here were each commercially available.

### Material:

| INCI Name | Trade Name | Supplier |
|---|---|---|
| LACTIC ACID | L(+)-LACTIC ACID | JUNGBUNZLAUER |
| | 90% HEAT STABLE | |
| | PERSONAL CARE | |
| | GRADE | |
| SALICYLIC ACID | SALICYLIC ACID | CELLMARK |
| | USP | |
| SODIUM LAURETH | KOPACOL N701 S | KENSING |
| SULFATE | RENU ULTRA/MB | PT MUSIM MAS |
| | MASPHATE ES70 | |
| | 1M LD RSPO MB | |
| DECYL GLUCOSIDE | PLANTACARE 2000 | BASF |
| | UP | |
| COCAMIDOPROPYL | AMONYL 675 SB | SEPPIC |
| HYDROXYSULTAINE | | |
| Sodium Hydroxide | SODIUM | PT. MULIA AGUNG |
| | HYDROXIDE 48% | CHEMINDO |
| TRIETHANOLAMINE | TRIETHANOL- | BASF |
| | AMINE CARE | |

### Example A

For different purposes, inventive Examples Ex.1-Ex.4 according to the present invention, as well as comparative Examples CE.1-CE.3, were prepared.

TABLE 1

| | Ex. 1 | Ex. 2 | Ex. 3 | Ex. 4 | CE. 1 | CE. 2 | CE. 3 |
|---|---|---|---|---|---|---|---|
| SODIUM LAURETH | 20% | 20% | 18% | 19.5% | 20% | 20% | 20% |
| SULFATE | | | | | | | |
| DECYL GLUCOSIDE | 0% | 1.0% | 1.0% | 0.5% | 0% | 0% | 1.0% |
| COCAMIDOPROPYL | 1.0% | 1.0% | 3.0% | 0.5% | 0% | 1.0% | 0% |
| HYDROXYSULTAINE | | | | | | | |
| COCOAMIDOPROPYL | 0% | 0% | 0% | 0% | 1.00% | 0% | 1.0% |
| BETAINE | | | | | | | |
| LACTIC ACID | 4% | 5.4% | 5.4% | 3.0% | 5.40% | 5.4% | 5.4% |
| SALICYLIC ACID | 1.5% | 1.5% | 1.5% | 1.0% | 1.50% | 1.5% | 1.5% |
| SODIUM | 0.17% | 0.31% | 0.31% | 0% | 0% | 1.8% | 0.31% |
| HYDROXIDE | | | | | | | |
| TRIETHANOLAMINE | 1.8% | 2% | 2% | 1.8% | 1.8% | 0% | 2% |
| WATER | QS to | QS to | QS to | QS to | QS to | QS to | QS to |
| | 100% | 100% | 100% | 100% | 100% | 100% | 100% |
| pH | 3.8 | 3.8 | 3.8 | 3.8 | 3.8 | 3.8 | 3.8 |

The above listed compositions were prepared according to known manufacturing method of field. Briefly, for example, the procedure of preparing the composition of Ex. 1 comprised the steps of:
1) adding acid of LACTIC ACID and SALICYLIC ACID, and TRIETHANOLAMINE, mix till dissolved;
2) adding surfactants (SODIUM LAURETH SULFATE, COCAMIDOPROPYL HYDROXYSULTAINE), mix till transparent; and
3) adjusting pH to the specification if needed, where if pH value was lower than 3.8 then TRIETHANOLAMINE was added till pH equal to 3.8; while if pH value was higher than 3.8 then lactic acid was added till pH equal to 3.8.

### Example B

The compositions of Example A were evaluated for the foaming performance according to the following steps:

For Foam Volume test protocol:
a. preparing 10 fH hardness water (111 mg CaCl₂) dissolved in 1 L DI water);
b. weighing 4 g tested composition and placing into kitchen blender;
c. weighing 76 g of prepared 10 fH water, to dilute the composition by 20 times;
d. turning on the kitchen blender and mixing for 1 minute;
e. quickly transferring the produced foam and liquid into a 500 ml graduate cylinder; and
f. reading the total volume.

Higher foam volume number means abundant foam.

For Stable Foam test protocol:
a. preparing 10 fH hardness water (111 mg CaCl₂) dissolved in 1 L DI water);
b. weighing 4 g tested shampoo and placing into kitchen blender;
c. weighing 76 g of prepared 10 fH water, to dilute shampoo into 20 times;
d. turning on the kitchen blender and mixing for 1 minute;
e. quickly transferring the produced foam and liquid into a 500 ml graduate cylinder; and
f. reading the Drainage height at 1 minute with a ruler

Smaller Drainage height number means more stable foam.

The foam smoothness of each shampoo was evaluated as follows:

Treating the hair with the tested shampoo:

A bundle of damaged hair was selected. The hair was combed and wet under running water, the tested shampoo was applied uniformly on the damaged hair from root to tail, the shampoo was gently massaged into the hair with two fingers from root to tail (without knotting) to foam the shampoo.

The foam was collected from the hair surface to hand, and the degree of non-resistance was judged when finger was sliding through the hair.

Ex.1 was used as the benchmark in parallel with the tested shampoo in above foam evaluation. 6 volunteers were recruited and the scores on foam smoothness of each tested shampoo were given by them.

The scoring standard for foam smoothness of each tested shampoo is as follows.
--: the tested shampoo is significantly inferior to the benchmark;
-: the tested shampoo is slightly inferior to the benchmark;
=: the tested shampoo is similar to the benchmark;
+: the tested shampoo is slightly better than the benchmark; and
++: the tested shampoo is obviously superior to the benchmark.

The results were provided in Table 2 below:

TABLE 2

| | Ex. 1 | Ex. 2 | Ex. 3 | Ex. 4 | CE. 1 | CE. 2 | CE. 3 |
|---|---|---|---|---|---|---|---|
| Foam Volume | 650 | 550 | 580 | 550 | 400 | 640 | 360 |
| Stable | 11.0 | 10.2 | 12.5 | 13.0 | 20.6 | 12.1 | 19.3 |
| Foam-Drainage | | | | | | | |
| height/mm | | | | | | | |
| Softness of Foam | = | ++ | ++ | + | = | −− | − |

It could be seen that the inventive Examples achieved desirable effects regarding all of volume, stability and softness of the foam. As compared, the comparative Examples led to one or more inferior effects.

1. A composition comprising:
A) a sulfur-containing anionic surfactant;
B) a betaine-derived amphoteric sultain surfactant;
C) an α-hydroxy acid;
D) a β-hydroxy acid; and
E) an alkanolamine.

2. The composition according to claim 1, wherein the component A) is at least one of a sulfate, a sulfonate, a sulfoacetate, or a sulfosuccinate.

3. The composition according to claim 1, wherein the component A) is present in an amount of from 1% to 50% by weight relative to the total weight of the composition.

4. The composition according to claim 1, wherein the component B) is selected from the group consisting of sulfobetaines.

5. The composition according to claim 1, wherein the component B) is present in an amount ranging from 0.1% to 20% by weight, preferably from 0.3% to 12% by weight, or preferably from 0.5% to 8% by weight, relative to the weight of the composition.

6. The composition according to claim 1, wherein the component C) is selected from the group consisting of citric acid, lactic acid, methyllactic acid, glucuronic acid, glycolic acid, pyruvic acid, 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytetradecanoic acid, 2-hydroxyhexadecanoic acid, 2-hydroxyoctadecanoic acid, 2-hydroxytetracosanoic acid, 2-hydroxyeicosanoic acid, mandelic acid, phenyllactic acid, gluconic acid, galacturonic acid, aleuritic acid, ribonic acid, tartronic acid, tartaric acid, malic acid, fumaric acid and their mixtures.

7. The composition according to claim 1, wherein the component C) is present in the composition in an amount of from 0.1 to 40% by weight relative to the total weight of the composition.

8. The composition according to claim 1, wherein the component D) is selected from the group consisting of salicylic acid, acetylsalicylic acid, capryloyl salicylic acid, 5-n-decanoylsalicylic acid, 5-n-dodecanoylsalicylic acid, 5-n-octylsalicylic acid, 5-n-heptyloxysalicylic acid, 4-n-heptyloxysalicylic acid, 5-tert-octylsalicylic acid, 3-tert-butyl-5-methylsalicylic acid, 3-tert-butyl-6-methylsalicylic acid, 3,5-diisopropylsalicylic acid, 5-butoxysalicylic acid, 5-octyloxysalicylic acid, 5-propanoylsalicylic acid, 5-n-hexadecanoylsalicylic acid, 5-n-oleoylsalicylic acid, 5-benzoylsalicylic acid, monovalent and divalent salts thereof, and mixtures thereof.

9. The composition according to claim 1, wherein the component D) is present in the composition in an amount of from 0.1 to 30% by weight relative to the total weight of the composition.

10. The composition according to claim 1, wherein the component E) is selected from the group consisting of monoethanolamine, diethanolamine, triethanolamine, monoisopropanolamine, diisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol and tris(hydroxymethylamino) methane; so as to result in a pH ranging from 2.5 to 6.5 for the composition.

11. The composition according to claim 1, further comprising a nonionic surfactant (F), selected from the group consisting of sugar esters of fatty acids, oxyalkylenated derivatives of sugar esters of fatty acids, sugar ethers of fatty alcohols, and mixtures thereof.

12. A method for cleansing keratin materials, comprising:
applying the composition according to claim 1 to the keratin material.

13. The composition according to claim 1, wherein the component B) is selected from the group consisting of (C8-C20) alkylsulfobetaines; alkyl (C8-C20) amidoalkyl (C1-C6) sulfobetaines and alkyl (C8-C20) amidoalkyl (C1-C6) hydroxyl sulfobetaines.

14. The composition according to claim 1, wherein the component A) is at least one of sodium laureth sulfate, sodium lauroyl methyl isethionate and sodium cocoyl isethionate.
